(19) United States
(12) Patent Application Publication
(10) Pub. No.: US 2004/0101539 A1
(43) Pub. Date: May 27, 2004

(54) Aqueous dispersion of low-melting organic solids

(76) Inventors: Ernst Engler, Grabs (CH); Gerhard Tiedtke, Gams (CH)

Correspondence Address: ROHM AND HAAS COMPANY, PATENT DEPARTMENT, 100 INDEPENDENCE MALL WEST, PHILADELPHIA, PA, 19106-2399, US

(21) Appl. No.: 10/702,422

(22) Filed: Nov. 5, 2003

Related U.S. Application Data
(60) Provisional application No. 60/449,894, filed on Feb. 25, 2003.
(60) Provisional application No. 60/428,414, filed on Nov. 22, 2002.

(51) Int. Cl. A61K 31/425; A61K 9/00

(52) U.S. Cl. 424/400; 514/372

(57) ABSTRACT

An aqueous composition comprising from 5% to 30% of at least one organic compound having a melting point in a range from 30° C. to 60° C. and water solubility at 25° C. of less than 0.5%, at least one inorganic filler, at least one surfactant and no more than 20% organic solvent. The composition is stable with regard to agglomeration and phase separation for at least three months at room temperature.

This invention relates generally to a stable aqueous dispersion of a low-melting, water-insoluble organic solid.

Biocidal active ingredients used for protection of coatings, such as exterior paints need to have low water solubility to prevent rapid leaching upon repeated exposure to rain. Adding such active ingredients into the matrix to be protected therefore can either be done by dosing the powder directly, or by preparing concentrated solutions in organic solvents or flowable dispersions. Handling of pure powders of such biologically active substances in technical manufacturing environments involves significant risk to employees of exposure to toxic dust. Such powders also have a wide variation in particle size which can limit the overall microbiological efficacy.

Solutions of the active ingredient in organic solvents are typically limited in concentration, and thus introduce considerable amounts of organic solvent (VOC) into the environment, and also are rather expensive. Flowable aqueous dispersions can be more concentrated than solutions, have less VOC and secure even distribution and uniform microbiological efficacy due to reduced particle size. They also minimize the risk of workforce exposure to the active ingredient and are commonly used in the industry. However, typically such dispersions start from a solvent solution to ensure even distribution of the active ingredient into the milling process, thus still introducing VOC, although less than solvent based solutions.

European Patent Application No. 1 060 667 discloses a method for producing a suspension of a crystalline solid in water. High-shear mixing was used to produce particles less than 5 microns in size. However, this reference does not teach the use of inorganic fillers. The problem addressed by this invention is to produce a stable aqueous dispersion of a low-melting organic compound with less than 20% organic solvent.

### STATEMENT OF THE INVENTION

This invention is directed to an aqueous composition comprising from 5% to 30% of at least one organic compound having a melting point in a range from 30° C. to 60° C. and water solubility at 25° C. of less than 0.5%, at least one inorganic filler, at least one surfactant and no more than 20% organic solvent. The composition is stable with regard to agglomeration and phase separation for at least three months at room temperature.

This invention is further directed to a method for producing an aqueous dispersion comprising from 5% to 30% of at least one organic compound having a melting point in a range from 30° C. to 60° C. and water solubility at 25° C. of less than 0.5%, at least one inorganic filler, at least one surfactant and no more than 20% organic solvent; said dispersion being stable with regard to agglomeration and phase separation for at least three months at room temperature. The method comprises combining in water the organic compound, the inorganic filler and the non-ionic surfactant, at a temperature from 0° C. to 35° C. with agitation to provide a substantially uniform dispersion.

### DETAILED DESCRIPTION OF THE INVENTION

An “organic compound” is a molecule containing no metal atoms, preferably a compound containing carbon and hydrogen atoms and other atoms selected from among nitrogen, oxygen, halogens, phosphorus, and sulfur. An “inorganic filler” is an inorganic material having a particle size less than 100 microns, and capable of remaining suspended in an aqueous dispersion. A “thickener” is a material which alters the rheological properties of an aqueous system to increase low-shear viscosity without greatly increasing viscosity at moderate shear rates, i.e., “pourability.”

Preferably, an organic compound in the composition of this invention has a melting point in the range from 35° C. to 50° C., more preferably from 35° C. to 45° C., and most preferably from 35° C. to 40° C. Preferably, an organic compound has water solubility at 25° C. of less than 0.05%. Preferably, the organic compound is a biocide, pesticide, or pharmaceutical; more preferably a biocide; and most preferably an isothiazolone biocide. Preferred isothiazolone biocides include 4,5-dichloro-2-octyl-3(2H)-isothiazolone (“DCOIT”) and n-butylbenzisothiazolone. The most preferred isothiazolone biocide is DCOIT. The water solubility of DCOIT is 2 ppm at 25° C., and its melting point is 40° C. Preferably, the composition contains at least 10% of at least one organic compound, more preferably at least 15%, more preferably at least 18%, and most preferably at least 20%. Preferably, the composition contains no more than 25% of the organic compound.

Preferably, the composition of this invention contains no more than 17% organic solvent, more preferably no more than 15%, more preferably no more than 10%, and most preferably no more than 7%. In a preferred embodiment of the invention, the composition has no more than 5% organic solvent, preferably no more than 2%, more preferably no more than 1%, and most preferably is substantially free of organic solvent. Preferably, organic solvents in the composition are di-glycol, tri-glycol or tetra-glycol solvents, or mixtures thereof, more preferably di-glycol or tri-glycol solvents, i.e., dimers or trimers of ethylene glycol or propylene glycol, or mixtures thereof. Di-glycol and tri-glycol solvents include, for example, triethylene glycol, dipropylene glycol, tripropylene glycol, and diethylene glycol. Preferably, the composition contains no more than 10% of organic solvents having a boiling point less than 250° C., more preferably no more than 8%, more preferably no more than 7%, and most preferably no more than 6%.

In one preferred embodiment of the invention, the composition contains at least one divalent copper salt. Suitable copper salts include, for example, copper dodecylbenzenesulfonate and copper EDTA complexes, including Na₂CuEDTA. Preferably, the composition contains at least 2% of at least one copper salt, more preferably at least 4%, and most preferably at least 4.5%. Preferably, the composition contains no more than 10% of copper salt(s), more preferably no more than 8%, and most preferably no more than 5.5%.

In the method of this invention, the organic solid(s), inorganic filler(s), surfactant(s), and optionally other ingredients, are combined with water with agitation, while the temperature is maintained from 0° C. to 35° C. Preferably, the temperature is maintained from 2° C. to 30° C., more preferably from 2° C. to 25° C., and most preferably from 2° C. to 20° C.

Preferably, at least 1% of at least one filler is present in the composition, more preferably at least 3%. Most preferably the amount of filler(s) is at least 6%. Preferably, the amount of filler(s) is no more than 20%, more preferably no more than 15%, and most preferably no more than 10%. Preferred fillers include, for example, calcium carbonate, silica, celite, talc, titanium dioxide, and clay, including kaolin.

Preferably, at least 0.1% of at least one surfactant is present in the composition, more preferably at least 0.2%, more preferably at least 0.5%. Most preferably the amount of surfactant(s) is at least 1%. Preferably, the amount of surfactant(s) is no more than 5%, more preferably no more than 3%, and most preferably no more than 2%. Preferred surfactants include, for example, non-ionic surfactants, including 2,4,7,9-tetramethyl-5-decyne-4,7-diol; C₉-C₁₁ alcohols ethoxylated with about 10 moles of ethylene oxide; and naphthalenesulfonic acid/formaldehyde polymers; and dodecylbenzenesulfonate metal salts.

Preferably, at least one thickener is added to the dispersion. Suitable thickeners include, for example, xanthan gum, silicic acid salts, acrylic acid polymers and copolymers, and colloidal metal silicates. Preferably, the total amount of thickener(s) is from 0.1% to 3%, more preferably from 0.5% to 2%, and most preferably from 1% to 2%.

Preferably, the particle size of the dispersion is reduced to less than 50 microns (μ), preferably less than 30μ. Particle size is determined either optically, with a scanning electron microscope, or using commercial particle size analyzers, e.g., those using laser light scattering to determine particle size. Typically, smaller particles provide greater stability to the dispersion. Preferably, the dispersion is stable with regard to concentration of the organic solid, as well as being stable with regard to agglomeration of the particles. In one embodiment of the invention, the particles are less than 50μ, preferably less than 45μ. In this embodiment, preferably at least 95% of the particles are in the range from 10μ to 45μ, more preferably from 15μ to 40μ.

The ingredients of the aqueous dispersion are agitated with any mixing equipment capable of producing a stable dispersion, for example, vacuum mixers, rotor/stator homogenizers, in-line emulsifiers, static mixers, piston homogenizers, ultrasonic homogenizers, high-speed jets or nozzles, and ball mills.

In one embodiment of the invention, the organic solid is dissolved in a solvent comprising at least one di-glycol or tri-glycol solvent prior to addition to other ingredients. Preferably, the amount of solvent is from 0.5 to 1 times the amount of the organic solid, by weight. Preferably, the solvent is a di-glycol or tri-glycol solvent, or a mixture thereof. In another embodiment, the organic solid is melted prior to addition to the other ingredients. In this embodiment, the organic solvent level preferably is no more than 5%. DCOIT melts at approximately 40° C.; preferably the molten DCOIT is added in substantially pure form without solvent.

The composition of this invention is stable with regard to agglomeration and phase separation for at least three months at room temperature, i.e., 20° C. to 25° C., and preferably for at least three months at 35° C.

### EXAMPLES

### Example 1

### Dispersion of DCOIT from DCOIT Solution

DCOIT (600.00 g) was crushed and dissolved by introducing the lumps into a mixture of 200.00 g dipropylene glycol (DPG) and 225.00 g triethylene glycol (TEG) at 40° C. in a 3000 ml round bottom flask on a rotary evaporator. Into this solution 15.60 g of Cu(OH)₂ and 105.30 g dodocylbenezenesulfonic acid (Marlon® AS 3) were added, forming CuDDBS in situ. The resulting solution was introduced into a mixture of 1727.10 g water, 30.00 g Surfinol™ TG/E surfactant (2,4,7,9-tetramethyl-5-decyne-4,7-diol; available from Chemische Fabrik Schweizerhall, Basel, Switzerland), 30.00 g Veegum™ thickener (silicic acid, aluminum magnesium salt; available from Christ Chemie Ltd., Reinach, Switzerland), 30.00 g TiO₂ and 12.00 g Kelzan™ CC thickener (xanthan gum; available from Staerkle & Nagler Ltd., Zurich, Switzerland) and pre-dispersed in 25.00 g DPG in a 6 L stainless steel beaker under gentle stirring (120 rpm). The resulting viscous liquid was pumped into a ball mill (Dynomill® KDL) filled to 80% with glass beads (1-1.5 mm particle size), running at 2500 rpm and cooled with EG/water of −5° C. at a rate such that the temperature of the dispersion exiting the mill did not exceed 25° C.; if necessary the process was stopped to allow cooling down to <20° C. The resulting dispersion, viscosity 80 KU, particle size (DIN 53 203) 18-25μ, [20% DCOIT, 7.50% DPG, 6.30% TEG, 4.03% CuDDBS stabilizer, 57.57% water and formulation aids to stabilize the dispersion (1% Surfinol™ TG/E, 1% Veegum™, 1% TiO₂, 1.2% TEG, 0.4% Kelzan™)] is stable with regard to viscosity changes and DCOIT concentration for at least 3 months at room temperature and 35° C.

### Example 2

### Dispersion of DCOIT from DCOIT Solution

DCOIT (800.00 g) was crushed and dissolved by introducing the lumps into a mixture of 250.00 g DPG and 300.00 g TEG at 40° C. in a 3000 ml round bottom flask on a rotary evaporator. The resulting solution was introduced into a mixture of 2056.00 g water, 200.00 g Na₂CuEDTA, 40.00 g Surfinol™ TG/E surfactant, 40.00 g Veegum™, 40.00 g TiO₂, 200.00 g kaolin (Kaolin FP 80, ground; minimum 52%<2μ; available from Gebr. Dorfner GmbH & Co., Hirschau, Germany) and 24.00 g Kelzan™ CC pre-dispersed in 50.00 g DPG in a 6 L stainless steel beaker under gentle stirring (120 rpm). The resulting viscous liquid was pumped into a ball mill (Dynomill® KDL) filled to 80% with glass beads (1-1.5 mm particle size), running at 2500 rpm and cooled with EG/water of −5° C. at a rate such that the temperature of the dispersion exiting the mill did not exceed 25° C.; if necessary the process was stopped to allow cooling down to <20° C. The resulting dispersion, viscosity 100 KU, particle size (DIN 53 203) 18-28μ, [20% DCOIT 7.5% DPG, 6.3% TEG, and 5.0% Na₂CuEDTA stabilizer in 51.4% water and formulation aids to stabilize the dispersion (1% Surfinol™ TG/E, 1% Veegum™, 1% TiO₂, 1.2% TEG, 0.6% Kelzan™, 5% kaolin)] is stable with regard to viscosity changes and DCOIT concentration for at least 3 months at room temperature and 35° C.

### Example 3

### Dispersion of DCOIT from Melted DCOIT

DCOIT (800.00 g) was crushed and melted at 50° C. The liquid DCOIT was poured slowly into a mixture of 2608.00 g water, 200.00 g Na₂CuEDTA, 40.00 g Surfinol™ TG/E surfactant, 40.00 g Veegum™, 40.00 g TiO₂, 200.00 g kaolin and 24.00 g Kelzan™ pre-dispersed in 48.00 g TEG in a 6 L stainless steel beaker under gentle stirring (140 rpm). The resulting viscous liquid was pumped into a ball mill (Dynomill® KDL) filled (80%) with glass beads (1-1.5 mm particle size), running at 2500 rpm and cooled with EG/water of −5° C. at a rate such that the temperature of the dispersion exiting the mill did not exceed 25° C.; if necessary the process was stopped to allow cooling down to <20° C. The resulting dispersion, viscosity 100 KU, particle size (DIN 53 203) 28-33μ, [20% DCOIT and 5% Na₂CuEDTA in 65.2% water and formulation aids to stabilize the dispersion (1% Surfinol™ TG/E, 1% Veegum™, 1% TiO₂, 1.2% TEG, 0.6% Kelzan™, 5% kaolin)] is stable with regard to viscosity changes and DCOIT concentration for at least 3 months at room temperature and 35° C.

1. An aqueous composition comprising from 5% to 30% of at least one organic compound having a melting point in a range from 30° C. to 60° C. and water solubility at 25° C. of less than 0.5%, at least one inorganic filler, at least one surfactant and no more than 20% organic solvent; said composition being stable with regard to agglomeration and phase separation for at least three months at room temperature.

2. The composition of claim 1 in which said at least one organic compound is at least one isothiazolone biocide having a melting point in a range from 35° C. to 45° c.

3. The composition of claim 2 containing from 15% to 30% of said at least one isothiazolone biocide, which is selected from 4,5-dichloro-2-octyl-3(2H)-isothiazolone and n-butylbenzisothiazolone.

4. The composition of claim 3 in which said at least one inorganic filler comprises titanium dioxide and kaolin, the composition contains no more than 17% organic solvent, and the composition further comprises at least one thickener and at least 2% of at least one copper salt.

5. The composition of claim 4 in which said at least one organic solvent comprises at least one solvent selected from di-glycol solvents and tri-glycol solvents.

6. A method for producing an aqueous dispersion comprising from 5% to 30% of at least one organic compound having a melting point in a range from 30° C. to 60° C. and water solubility at 25° C. of less than 0.5%, at least one inorganic filler, at least one surfactant and no more than 20% organic solvent; said dispersion being stable with regard to agglomeration and phase separation for at least three months at room temperature; said method comprising combining in water said organic compound, the inorganic filler and the surfactant, at a temperature from 0° C. to 35° C. with agitation to provide a substantially uniform dispersion.

7. The method of claim 6 in which the dispersion contains from 15% to 30% of said at least one isothiazolone biocide, which is selected from 4,5-dichloro-2-octyl-3(2H)-isothiazolone and n-butylbenzisothiazolone, and the temperature is maintained from 2° C. to 25° C.

8. The method of claim 7 in which said at least one inorganic filler comprises titanium dioxide and kaolin, the dispersion contains no more than 17% organic solvent, and in which the dispersion further comprises at least one thickener and at least 2% of at least one copper salt.

9. The method of claim 8 in which the organic compound is 4,5-dichloro-2-octyl-3(2H)-isothiazolone which is dissolved in a solvent comprising at least one solvent selected from di-glycol solvents and tri-glycol solvents prior to addition to other ingredients.

10. The method of claim 8 in which the organic compound is 4,5-dichloro-2-octyl-3(2H)-isothiazolone which is melted prior to addition to other ingredients.
